(19) United States
(12) Patent Application Publication
(10) Pub. No.: US 2003/0195415 A1
(43) Pub. Date: Oct. 16, 2003

(54) Device, system and method for accoustic in-vivo measuring

(76) Inventor: Gavriel J. Iddan, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/365,612

(22) Filed: Feb. 13, 2003

Related U.S. Application Data
(60) Provisional application No. 60/356,168, filed on Feb. 14, 2002.

(51) Int. Cl. A61B 5/05

(52) U.S. Cl. 600/424

(57) ABSTRACT

A system and method senses an in-vivo lumen using ultrasonic elements typically arranged in a ring or other similar structure. Position information may be collected. A set of reflectance data may be collected and used to form an image or representation of the lumen. In one example, the data is collected by an in-vivo autonomous capsule. Additionally, ultrasonic elements may be arranged in order to receive a mechanical characteristic of the tissue (e.g., acoustic impedance) rather than an image or representation.

### PRIOR PROVISIONAL PATENT APPLICATION

The present application claims benefit from prior provisional patent application serial No. 60/356,168 filed on Feb. 14, 2002 and entitled “ACCOUSTIC IN-VIVO MEASURING SYSTEM”, incorporated herein by reference in its entirety.

### FIELD OF THE INVENTION

The present invention relates to an in vivo device, system and method for providing information on a body lumen; more specifically, to an in vivo device, system and method for producing an image or representation of an in-vivo lumen.

### BACKGROUND OF THE INVENTION

Devices and methods for performing in-vivo imaging of passages or cavities within a body are known in the art. Such devices may include, inter alia, various endoscopic imaging systems and devices for performing imaging in various internal body cavities.

Typical current in-vivo imaging devices use light or other electromagnetic energy to form images. Images based on light or other electromagnetic energy may not provide information on, for example, features or structures obscured by the contents of the gastrointestinal (GI) tract or beyond or behind the surface of the lumen being imaged. A medical practitioner may desire to image such structures or features.

Further, when imaging the GI tract, a thorough cleaning may be required beforehand. In particular, the colon may be filled with matter such feces, while other parts of the GI tract may be filled with more liquid which is more transparent. However, various parts of the GI tract may also be filled with more opaque matter. Such cleaning may be involved and uncomfortable, for example requiring a multi day liquid diet or low residue diet, or the use of special cleaning agents such as laxatives.

Therefore, there is a need to provide images or representations of, or information on, in-vivo lumens, typically without a prior cleaning, and including structures or features that are hidden, beneath or behind contents of the lumen or the surface of the lumen.

### SUMMARY OF THE INVENTION

In one embodiment, a system and method senses an in-vivo lumen using ultrasonic elements typically arranged in a ring or other similar structure. Location and/or orientation information may be collected. A set of reflectance data may be collected and used to form an image or representation of the lumen. In one example, the data is collected by an in-vivo autonomous capsule. Additionally, ultrasonic elements may be arranged in order to receive a mechanical characteristic of the tissue (e.g., acoustic impedance) rather than an image or representation.

### BRIEF DESCRIPTION OF THE DRAWINGS

The present invention will be understood and appreciated more fully from the following detailed description taken in conjunction with the drawings in which:

FIG. 1 shows a schematic diagram of an in vivo imaging system according to one embodiment of the present invention;

FIG. 2 depicts an ultrasonic element extending from the wall of a device, according to one embodiment of the present invention;

FIG. 3 depicts an activation pattern of a set of ultrasonic elements in an in-vivo device according to an embodiment of the present invention;

FIG. 4 is a depiction of a device within a body lumen according to one embodiment of the present invention;

FIG. 5 depicts a series of graphic representations based on ultrasonic data, according to an embodiment of the present invention; and

FIG. 6 depicts a representation produced by a system and method according to an embodiment of the present invention.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Embodiments of the system and method of the present invention are typically used in conjunction with an in-vivo sensing system or device. Examples of in-vivo sensing devices providing image data are provided in embodiments described in U.S. Pat. No. 5,604,531 to Iddan et al. and/or in International Application number WO 01/65995 entitled “A Device And System For In Vivo Imaging”, published on Sep. 13, 2001, both of which are hereby incorporated by reference in their entirety. Such embodiments generally use light or electromagnetic radiation to provide images, while various embodiments of the present invention use ultrasonic energy to provide such images. Typically, a device according to the present invention need not include video imaging capability, although it is within the scope of the present invention to include video or other types of imaging capability. However, certain features of the embodiments described in U.S. Pat. No. 5,604,531 and/for International Application WO 01/65995 may be used in embodiments of the present invention. In addition, the device, system and method according to the present invention may be used with any device, system and method sensing a body lumen or cavity.

While one typical use of embodiments of the present invention is imaging or examining the colon, other parts of the GI tract, and other lumens, may be imaged or examined.

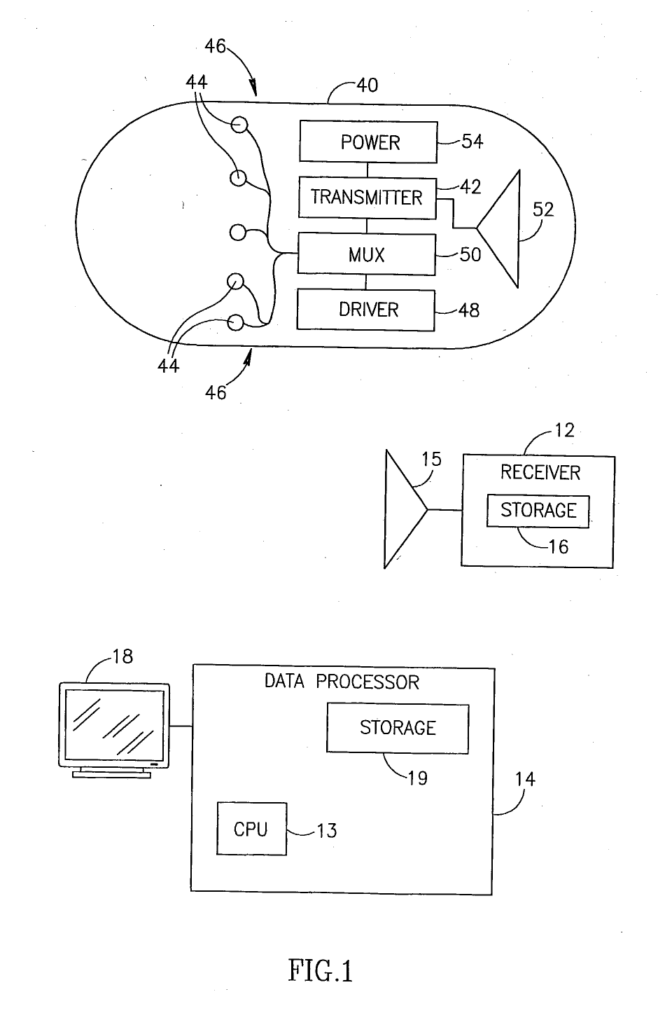

Reference is made to FIG. 1, which shows a schematic diagram of an in vivo imaging system according to one embodiment of the present invention. Referring to FIG. 1, device 40 is an in-vivo sensing device. In a typical embodiment, a device 40 is a swallowable capsule which is typically autonomous and typically ingestible; however, other shapes and configurations may be used. Elements of device 40 may be, for example, similar to embodiments described in U.S. Pat. No. 5,604,531 and/or International application WO 01/65995, described above. However, the device may be any sort of in-vivo sensor device and may have other configurations. A vehicle other than a capsule may be used, such as a device having the shape of a sphere or an endoscope.

In one embodiment of the present invention, device 40 includes a set of ultrasonic elements 44 (where set can include one element), an ultrasonic driver 48, a multiplexer 50, and a transmitter 42, for transmitting information to a receiving device. Typically, multiplexer 50 interfaces between the ultrasonic elements 44, the ultrasonic driver 48, and transmitter 42. Ultrasonic driver 48 drives the ultrasonic elements 44. Multiplexer 50 connects ultrasonic driver 48 and transmitter 42 to certain of the ultrasonic elements 44 to produce the required ultrasonic activation patterns. When the ultrasonic elements 44 act as ultrasonic receivers, the multiplexer 50 connects the reception elements to the transmitter 42 accordingly. Multiplexer 50 may include a processing element (not shown) for determining the required activation patterns of the ultrasonic elements. In one embodiment, an ultrasonic element 44 transmits energy, is switched off, and receives energy back. The phasing and control of the receipt of energy may be patterned after the phasing and control of the transmission. Other patterns and methods of control are possible. Connections between components may be other than as shown.

Typically, the ultrasonic elements 44 include piezoelectric materials which can both send and receive ultrasonic energy (e.g., a monostatic unit). In alternate embodiments bistatic units may be used, having separate units for transmission and for reception. Other sets or arrangements of elements may be included, and devices having a configuration other than shown in U.S. Pat. No. 5,604,531 to Iddan and/or or International Application WO 01/65995 may be used. For example, a multiplexer may be omitted.

Typically, the ultrasonic elements 44 are arranged in at least one circumferential ring 46 around the circumference of the device 40. Multiple rings 46 or a single ring 46 may be used. Viewing the device 40 in cross section, the ultrasonic elements 44 are in one embodiment arranged in ring 46 around the side surface of device 40; the elements may extend slightly from the device 40. Typically, a radial pattern of ultrasonic energy is produced. Other arrangements or arrays of ultrasonic elements may be used, and other numbers of arrays may be used. For example, a ring need not be used. Furthermore, the ring need not be in the shape of an exact circle, and need not have elements regularly spaced. The ultrasonic elements 44 may be arranged to be parallel with the axis of the device 40, or lengthwise, rather than perpendicular to the axis. In another embodiment, a single transducer at the head of the device 40 or one end of the device 40 may send out ultrasonic energy in field of, for example, 180 degrees, and receive an echo to measure acoustical impedance. The device 40 may have other shapes or configurations, with other arrangements of ultrasonic devices. Ultrasonic elements 44 may be energized one by one or in sets (e.g., sequentially), the entire array may be energized simultaneously, or other patterns or methods of activation may be used.

The transmitter 42 is typically an ultra low power radio frequency (RF) transmitter with high bandwidth input, possibly provided in chip scale packaging. The transmitter 42 may transmit data, such as ultrasonic reflectance data, via one or more antenna(s) 52. The transmitter typically includes circuitry and functionality for controlling the device 40, and for controlling the output and collecting the input of ultrasonic elements 44. Typically, the device 40 includes a power source 54, such as one or more batteries. For example, the power source 54 may include silver oxide batteries, lithium batteries, or other electrochemical cells having a high energy density, or the like. Other power sources may be used.

Other components and sets of components may be used. For example, the power source may be an external power source transmitting power to the device 40, and a controller separate from the transmitter 42 may be used.

Preferably, located outside the patient's body in one or more locations, are a receiver 12, preferably including an antenna or antenna array 15, for receiving data from device 40, a receiver storage unit 16, for storing data, a data processor 14, a data processor storage unit 19, and an image monitor 18, for displaying, inter alia, an image or representation of an in-vivo lumen transmitted by the device 40 and recorded by the receiver 12. Typically, the receiver 12 and receiver storage unit 16 are small and portable, and are worn on the patient's body during recording of the data. Preferably, data processor 14, data processor storage unit 19 and monitor 18 are part of a personal computer or workstation, which includes standard components such as a processor 13, a memory (e.g., storage 19, or other memory), a disk drive, and input-output devices, although alternate configurations are possible. In alternate embodiments, the data reception and storage components may be of another configuration. In addition, a data decompression module for decompressing data may also be included.

The receiving and recording components may be, for example, similar to embodiments described in the above-mentioned U.S. Pat. No. 5,604,531 and/or WO 01/65995. However, the receiving and recording components may be of other configurations.

The receiver 12 may also include a transmitter which can transmit to the device 40, for example, instructions regarding, for example, beam shaping and frequency used by the ultrasonic elements 44.

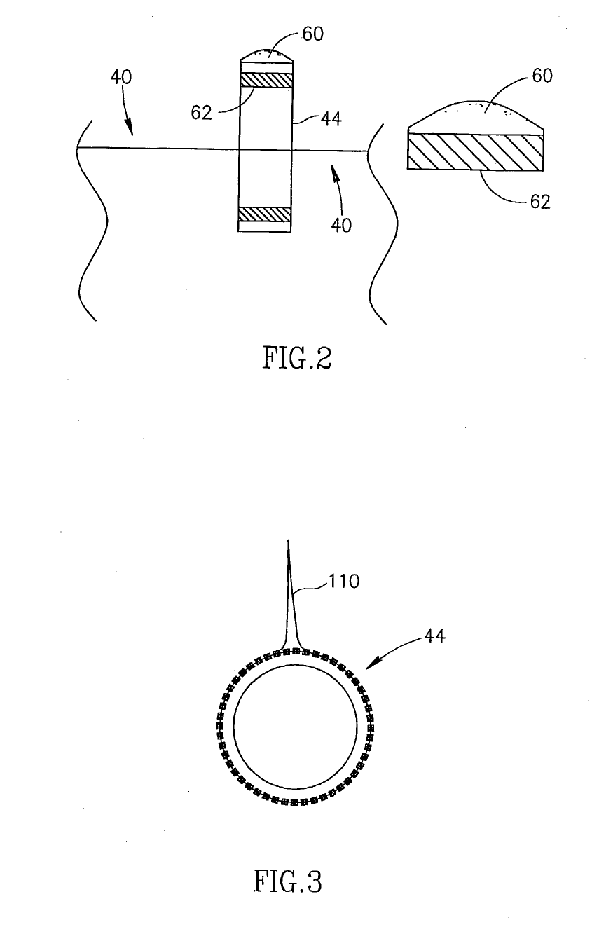

FIG. 2 depicts an ultrasonic element 44 extending from the wall 40′ of the device 40, according to one embodiment of the present invention. In other embodiments, ultrasonic elements may be flush with or recessed from the device wall 40′. Ultrasonic element 44 typically has mounted on it an ultrasonic lens 60 as known in the art. Other shapes or types of lenses may be used. Typically, a matching structure 62, such as an annular matching ring (or other structure), is placed between the ultrasonic element 44 and ultrasonic lens 60. Ultrasonic element 44 is typically a piezo element, and may act as an ultrasonic receiver, but may be of other constructions, and may lack reception capability. Other shapes and types of ultrasonic elements, having other components, may be used.

Each ultrasonic element 44 is typically a piezo element, including piezoelectric materials, with a dome or other shaped ultrasonic lens shaping ultrasonic energy into, for example, a point; typically the energy extends in an axial direction. Typically the ultrasonic elements 44 can both send and receive ultrasonic energy, but separate units for transmission and for reception may be used.

By using a set of ultrasonic elements 44 arranged in a ring 46 and activating certain of the ultrasonic elements 44 in a phased or patterned manner, the beam may be focused and directed. Typically, the beam is moved in a radial manner around the circumference of the device 40, typically perpendicular to the axis of the device 40, although other beam or ultrasonic patterns are possible. Thus a moving pattern of ultrasonic energy is created. Such movement is typically performed under the control of a controller (e.g., transmitter 42), by activating successive ultrasonic elements 44 or sets of ultrasonic elements 44 (when used herein set can include one unit). Transmitter 42 may include beam shaping and other functionality for controlling ultrasonic elements 44. Such functionality may be partially or completely implemented in multiplexer 50, or alternately in a separate unit (e.g., an ultrasonic controller). Further, the transmitter 42 may include receiver capabilities for, for example, receiving control functions or commands from an external transmitter (e.g., receiver 12, which may include transmission ability)

Alternate embodiments may not require focusing capabilities. In alternate embodiments, ultrasonic reflectance data may be recorded to measure, for example, an average mechanical tissue compliance, ultrasonic (acoustic) impedance along the lumen being imaged, etc. Such data maybe received by device 40 and transmitted as described elsewhere. Such data may be displayed in a manner other than an image or representation of the lumen; for example a graph may be presented.

FIG. 3 depicts an activation pattern of a set of ultrasonic elements in an in-vivo device, according to an embodiment of the present invention. Referring to FIG. 3, a beam 110, in this case a fine “pencil” beam, of ultrasonic energy may be created. Other shapes of beams may be used. In one embodiment, a set of ultrasonic elements 44 may be activated out of phase so that the some of all the waves create a pencil or other shaped beam. Acting simultaneously, several ultrasonic elements 44 (e.g., four or five) may create tangential focusing. The beam 110 may be rotated or scanned by sequentially activating subsets of ultrasonic elements 44. For example, elements 1-4, 2-5, 3-6, etc. may be sequentially activated. Overlapping elements may be reactivated in different phases to produce a desired beam. Thus a rotating pencil beam that can scan a radius may be created. In one embodiment, about 20 ultrasonic elements 44 are used, but other numbers may be used. The beam 110 can rotate possibly thousands of times per second; other rates may be used. While in FIG. 3 four or five ultrasonic elements 44 are activated at once, other numbers of ultrasonic elements 44 may be activated at one time. Other methods of altering the beam, instead of rotation, may be used. Typically, the control of the ultrasonic elements 44 is provided by multiplexer 50. Control of ultrasonic elements 44 may be based in other elements, such as transmitter 42. Known methods of controlling the activation of the ultrasonic elements 44 may be used.

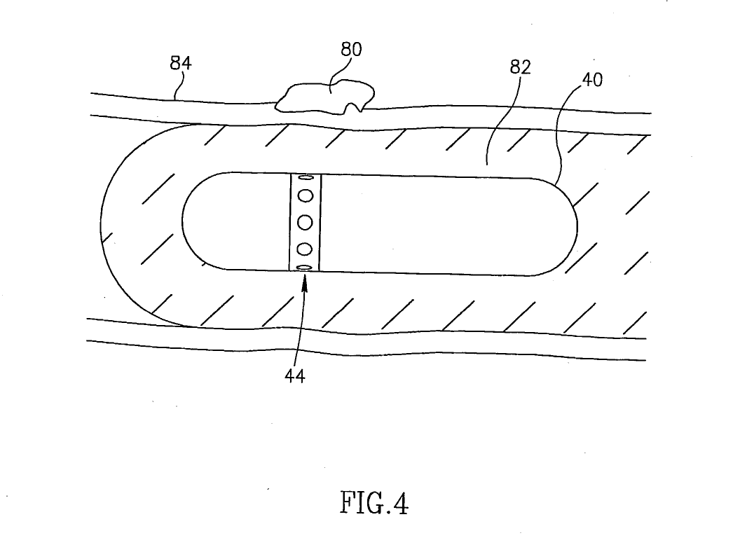

Typically, the ultrasonic energy reflected from the surrounding tissue or other objects activates ultrasonic elements 44, thus creating electrical signals. The electrical signals generated by the activated ultrasonic elements 44 may be temporarily stored and/or transmitted through transmitter 42 to receiver 12. Such signals may be used, as described below, to create an image or representation of the lumen. FIG. 4 is a depiction of the device 40 within a body lumen 84, according to one embodiment of the present invention. Referring to FIG. 4, the ultrasonic elements 44 transmit ultrasonic energy and receive reflectance information from various objects, such as object 80. In one in-use situation, the device 40 may be surrounded my material 82, such as liquid, stomach content or feces, but need not be. The device 40 may be in contact with the walls of the lumen 84. The device may be a shape or configuration other than that depicted, such as a sphere, a part of an endoscope, needle, catheter etc.

In a typical embodiment, position (e.g., location and/or orientation) information for the device 40 are determined. In alternate embodiments, position information need not be used. Typically, in applications involving the colon, orientation information is desirable, but need not be used; further, orientation information may be used in other applications.

Position data may include location and/or orientation data. Position determining elements may be included within the device (e.g., magnetic coils, a transmitter or antenna) and/or may be external to the device. In one embodiment, location determining elements can be part of the transmitter and/or antenna transmitting other data.

In a typical embodiment, location detection methods such as those discussed in United States patent application publication number US-2002-0173718-A1, filed May 20, 2002, entitled “Array System and Method For Locating an In-Vivo Signal Source,” assigned to the assignee of the present invention, and incorporated herein by reference, may be used.

Other location and/or orientation detection methods may be used. In one embodiment, the orientation information includes three Euler angles or quaternion parameters; other orientation information may be used, for example based on 5 or 6 location/orientation parameters (other numbers may be used). Location and orientation information may be determined by, for example, including two or more transmitting antennas in the above devices, each with a different wavelength, or by detecting the location and orientation using a magnetic method. Methods such as those using ultrasound transceivers or monitors that include, for example, three magnetic coils that receive and transmit positional signals relative to an external constant magnetic field may be used. A GPS or GPS like system may be used; for example a system using transmission from 3 or more stations. If a phase and frequency is used which is high enough (e.g., 300 MHz), a resolution of 1 mm is possible. Other GPS or GPS like systems may be used.

In one embodiment, a transceiver within the device includes, for example, three electrodes, coils or transponders that receive signals (e.g., electromagnetic signals) transmitted from an external source. The external source includes, for example, three transmitters (e.g., electromagnetic transmitters) at a fixed position in an external reference frame that transmit, for example, three distinguishable electromagnetic radiations (such as at different frequencies, or different time slots). The electrodes, coils or transponders receive signals corresponding to the different electromagnetic radiations at a plurality of times, each of the signals including components of at least one of the different radiations. The position and the orientation of the device can be determined from the data received from electrodes, coils or transponders. The electrodes, coils or transponders form signals that include the components of the signal received by the each electrode from the three transmitters.

Calculations for determining the in vivo position and orientation of objects may be carried out on suitable computational or processing devices, for example using data processor 14 and the appropriate software. Such calculations may be any of those known methods described above. For example, data which may aid in location and/or orientation determination is transmitted via, for example, transmitter 42, received by receiver 12, and downloaded to data processor 14. Alternately, processing capability within the device can determine a position within the reference frame, and this position information may be transmitted via transmitter 42 to be downloaded to data processor 14.

Of course, other location and/or orientation determining methods may be used.

Typically, data processor 14 collects information including the position of the device 40, the orientation of the device 40, and the ultrasonic information collected by the device 40 at each position. Note in alternate embodiments, orientation and/or position information may be omitted. In one embodiment, this information may be used to create a representation of the lumen (e.g., the GI tract) which is being examined.

In one embodiment, as the device 40 traverses a lumen, ultrasonic elements 44 (under the control of the multiplexer 50 and/or transmitter 42) emit ultrasonic energy- and record ultrasonic reflectance data (other elements can perform such recording). This reflectance data is transmitted by transmitter 42 to, for example, the receiver 12, and is eventually passed to data processor 14. Typically, position and possibly orientation data is also passed to data processor 14. Data processor 14, as discussed below, creates from the reflectance data and possibly location data (and possibly other data) an image or representation of an in-vivo lumen, typically displayed on monitor 18. Other sequences of operation, and other components, may be used, and other data may be passed.

In one embodiment, at each of a set of locations along the lumen (e.g., several thousands or tens of thousands of locations, although other numbers may be used) a set of ultrasonic reflectance information may be determined by the device 40 and received by the processor 14. In one embodiment, each set of ultrasonic information is a ring of ultrasonic reflectances recorded by, for example, one or more arrays of ultrasonic elements 44 on device 40, such as ring 46 (FIG. 1), or other sets of ultrasonic elements. Other sets of ultrasonic information may be recorded.

Typically, position information is recorded or calculated for each such location, and thus for each set of ultrasonic reflectance information, information on the position is also recorded or associated. For each location along the lumen, the ultrasonic reflectance information is used to produce a portion of an image or representation of the lumen. These image portions are combined, and are located in an overall image or representation, using the position information associated with each set of ultrasonic reflectance information. In alternate embodiments other methods of processing, using, or conveying ultrasonic data may be used. For example, diagnoses may be created, without providing images to a user.

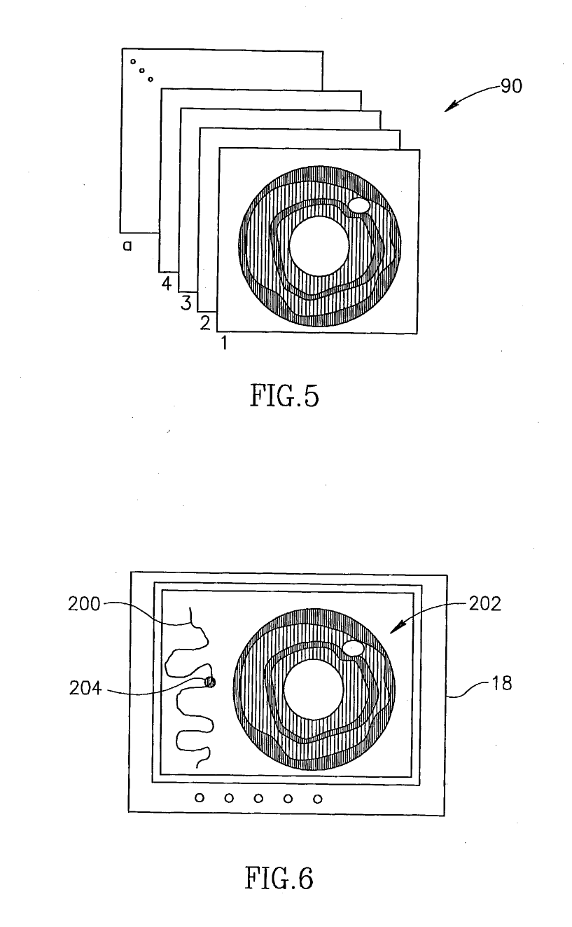

FIG. 5 depicts a series of graphic representations based on ultrasonic data, according to an embodiment of the present invention. Referring to FIG. 5, ultrasonic representations 90 (numbered 1-a) each are created from a set of ultrasonic reflectances (where set can include one element). Typically, the ultrasonic reflectances are recorded from a ring pattern of ultrasonic beams, but other patterns or types of ultrasonic output may be used. Typically, each representation 90 corresponds to a position within the body lumen being sensed, and these positions may be associated with the representations 90. Each representation 90 may be, for example, a “slice” image or representation created by a ring of ultrasonic reflectances. The data processor 14 (or another element) may create an image or other representation from each slice. The slices may be combined to create a view or representation of the lumen; typically, the position of each slice and the position (e.g., orientation and/or location) of the capsule when each slice was recorded are known and such information is combined with the image data to create an overall representation. Each slice need not be a flat, two dimensional representation; the representation may extend outward from the plane of the slice.

The acoustical image portions, and thus the overall acoustical image or representation, may include information not detectable by visible light, for example, it may allow a lumen wall (e.g. a colon wall) filled with opaque content (e.g. feces) to be imaged and/or a lumen containing numerous indentations (e.g. a colon) where the corners around the indentations cannot sufficiently lighted, to be imaged.

The image portions, and thus the overall image or representation, may include information not detectable by visible light; for example layers or objects beyond the inner surface of the lumen (e.g., a tumor, etc). Typically, each layer or object reflects ultrasonic energy in a different time sequence and with a different intensity. The device 40 may not be coaxial with the lumen.

In one embodiment, the data processor 14 displays on monitor 18 a representation such as that shown in FIG. 6. Referring to FIG. 6, monitor 18 displays a path representation 200 of the lumen through which the device 40 travels, a “slice” or two dimensional ultrasonic image of the lumen, typically in a plane perpendicular to the path of the device 40, and a position indication 204 of the device 40 along the path representation 200 corresponding to the image 202. Typically, the path representation 200 conforms in shape to the actual path of the device 40 through the lumen. Since the monitor is typically two dimensional, and the path of the device 40 is typically three dimensional, the path representation 200 may be two dimensional, or may be displayed using techniques that include three dimensional information to the two dimensional image. For example, shading or coloring may indicate three dimensional aspects; other techniques may be used. The image is typically a moving image, and thus as the position indication 204 moves along the path representation 200 the two dimensional image 202 changes accordingly. Controls such as freeze frame, speed and direction controls, may be included. Typically, the images are viewed after the device 40 has traversed the body lumen, although real time or near real time viewing may be performed. Location and orientation information may be used in the case that guiding or moving the capsule through the lumen is desired.

In alternate embodiments, other image representations may be created, and other sorts of analyses may be performed on the collected data. For example, a three dimensional (or simulated three dimensional) image of the GI tract and its surrounding tissues may be created. The various layers and objects depicted may be indicated by shadings or colors.

In the case of imaging of the GI tract, the GI tract may not have to be “cleaned” before the use of a device 40 according to one embodiment of the present invention. In alternate embodiments, a prior cleaning may be performed. Typically, gas such as air pockets interferes with the ultrasonic beams. When the device 40 is in the small intestine, there is typically liquid surrounding the device 40 (occasional gas bubbles also exist), and sometimes the device 40 touches the lumen wall, and thus typically gas produces few problems. When traversing the large intestine, the device 40 may be typically small with respect to the diameter of the lumen. However, the large intestine is typically full of content (e.g., feces) which is largely liquid and “soft” solid. Typically, content such as liquid, soft solids, etc., provide an impedance matching material for the ultrasonic energy. The ultrasound energy may penetrate beyond the content. Typically, the processor reconstructing the image of the lumen (e.g., data processor 14) is able to interpret certain reflections as air, in which case reconstruction may not take place for that portion and a blank or “air” spot may be indicated. This is typically indicated by relatively large reflection close to the device 40. The processor may also interpret and indicate to the user certain reflections as “liquid.”

In another embodiment, a single transducer at the head of the device 40 or one end of the device 40 may send out ultrasonic energy in field of, for example, 180 degrees, and receive an echo to measure acoustical impedance.

In certain embodiments, multiple methods of collecting sensing data may be used. For example, one or more of a single transducer, a set of transducers, and/or an optical imager may be used, and one modality may augment another. For example, a graph or other representation may be created of acoustical impedance to gather information which can be used to mark portions of an associated image stream as significant. A plurality of ultrasonic transmitters may be used with such an embodiment, at the tip of the device 40, along a circumference, or in other positions.

In alternate embodiments, multiple images may be acquired using multiple ultrasonic frequencies for the same locations in the lumen.

In one embodiment of the system and method of the present invention, a device may measure an average (e.g., typical) mechanical compliance of slices of tissue (e.g., using ultrasonic or acoustical impedance). In such an embodiment, the measured values may be, for example, presented on a graph. A possible pathology may be observed as, for example, a deviation from the typical values of the acoustic impedance. A multiple frequency (e.g. f1 and f2) graph may be presented in order to strengthen the single frequency findings.

It will be appreciated by persons skilled in the art that the present invention is not limited by what has been particularly shown and described herein above. Rather, the scope of the invention is defined by the claims that follow:

1. An in-vivo sensing device comprising:
a set of ultrasonic elements arranged in a ring; and
a controller capable of causing the set of ultrasonic elements to generate a pattern of ultrasonic energy.

2. The device of claim 1 comprising a transmitter.

3. The device of claim 1, comprising a plurality of rings of ultrasonic elements.

4. The device of claim 1 comprising a battery.

5. The device of claim 1 wherein the pattern is moving.

6. The device of claim 1 wherein the pattern moves in a ring.

7. The device of claim 1 comprising a set of ultrasonic receivers.

8. The device of claim 1 wherein each of the set of ultrasonic elements includes an ultrasonic receiver.

9. The device of claim 1, comprising a position determining element.

10. The device of claim 1, wherein the device is autonomous.

11. The device of claim 1, wherein the device is a swallowable capsule.

12. An in-vivo sensing platform comprising:
a processor capable of receiving a set of ultrasonic data, the set of ultrasonic data representing a body lumen, the set of ultrasonic data including reflectances received by a ring of ultrasonic receivers, and, in response, generating a representation of the body lumen.

13. The platform of claim 12 comprising a memory.

14. The platform of claim 12, wherein the ultrasonic data is collected by a swallowable capsule.

15. The platform of claim 12, wherein the data is received from a sensor device, and wherein processor is further capable of accepting data describing the position of a sensor device within the body lumen.

16. The platform of claim 12, wherein the data is received from a sensor device, and wherein processor is further capable of accepting data describing the location of a sensor device within the body lumen.

17. The platform of claim 16, wherein the processor is capable of combining the data describing the location with the set of ultrasonic data to generate the representation.

18. The platform of claim 12, wherein the data is received from a sensor device, and wherein processor is further capable of determining the location of a sensor device within the body lumen.

19. A method of creating a representation of a body lumen, the method comprising:
accepting a set of ultrasonic data, the ultrasonic data including sets of radial reflectances;
accepting a set of position data, each ultrasonic datum corresponding to an ultrasonic datum;
creating a set of images from the set of ultrasonic data; and
associating each image with a position datum.

20. The method of claim 19, wherein the ultrasonic data is collected by a swallowable capsule.

21. The method of claim 19, comprising generating a representation from the data.

22. An in-vivo sensing device comprising:
a ring of ultrasonic elements;
a position determining element; and
a controller.

23. An in-vivo sensing device comprising:
a set of ultrasonic element means for transmitting ultrasonic energy; and
a controller means for controlling the set of ultrasonic elements to generate a pattern of ultrasonic energy.

24. An in-vivo imaging platform comprising:
a processor means for receiving a set of ultrasonic data, the set of ultrasonic data representing a body lumen, the set of ultrasonic data including reflectances received by a ring of ultrasonic receivers, and, in response, generating a representation of the body lumen.

25. An in-vivo sensing platform comprising:
a processor capable of receiving a set of ultrasonic data and a set of position data, the set of ultrasonic data representing a gastrointestinal tract, the set of ultrasonic data including reflectances received by a ring of ultrasonic receivers in an autonomous capsule, and, in response, generating a representation of the body lumen.

26. A method of creating an image of a gastrointestinal track, the method comprising:
accepting a set of ultrasonic data collected by an autonomous capsule, the ultrasonic data including sets of radial reflectances;
accepting a set of position data, each ultrasonic datum corresponding to an ultrasonic datum;
creating a set of images from the set of ultrasonic data, each image corresponding to a section of the gastrointestinal tract; and
associating each image with a position datum.
